(19) United States
(12) Patent Application Publication
(10) Pub. No.: US 2003/0049839 A1
(43) Pub. Date: Mar. 13, 2003

(54) Transparent multi-channel cell scaffold that creates a cellular and/or molecular gradient

(75) Inventors: Mario I. Romero-Ortega, Carrollton, TX (US); Mauricio R. Delgado-Ayala, Dallas, TX (US); Pedro J. Galvan, Mission, TX (US); Hua Liu, Richardson, TX (US)

Correspondence Address: VINSON & ELKINS, L.L.P., 1001 FANNIN STREET, 2300 FIRST CITY TOWER, HOUSTON, TX, 77002-6760, US

(73) Assignee: The University of Texas System

(21) Appl. No.: 10/209,966

(22) Filed: Aug. 1, 2002

Related U.S. Application Data
(60) Provisional application No. 60/309,356, filed on Aug. 1, 2001.

(51) Int. Cl. C12N 5/00

(52) U.S. Cl. 435/397; 435/303.1

(57) ABSTRACT

A cell growth scaffold provides individual cell growth channels in a transparent body for microscopic observation of cells during growth.

This application claims benefit of U.S. Provisional Application No. 60/309,356, filed Aug. 1, 2001, now abandoned.

### BACKGROUND OF THE INVENTION

The issue of nerve repair is of great interest in both its clinical and basic science aspects. Injury to central (brain and spinal cord) or peripheral nerves could lead to either partial or complete loss of sensory and/or motor functions. Damage to the spinal cord often results in tissue loss and cavity formation at the site of injury, and although currently there is no treatment for this condition, it has been demonstrated experimentally that recovery of neurological function can be achieved by implanting a nerve-growth promoting conduit at the injury site. On the other hand, peripheral nerve injuries in which a nerve defect or gap is created, require the reestablishment of appropriate terminal contacts through direct anastomosis or auto-graft repair procedures. However, functional recovery is seldom achieved. A nerve injury differs from most other types of tissue injury. For example, recovery requires more than just a local repair process. Transection of axons impacts the whole length of the neuron, and the repair process involves outgrowth of neurites over long distances. In addition, in contrast to most other injuries of the body, nerve injury has immediate functional consequences, as expressed in abnormal sensibility and deficient motor function. During the last 25 years, knowledge and understanding of biological mechanisms of nerve injury, growth and regeneration have increased enormously. However, this has not resulted in a corresponding development in the clinical field leading to improved results from nerve repair.

The clinical results of peripheral nerve repair remain disappointing despite the use of several approaches for promoting nerve regeneration. Untreated gap lesions in peripheral nerve do not regenerate functioning tissue. Although axons undergo elongation in an untreated lesion, there is no directional regulation to direct the growth toward the distal stump, and the growing axons thus form a tangled web of unorganized neuroma. The current gold standard for nerve repair remains the autologous nerve graft, which requires that a healthy nerve be sacrificed and harvested. There are numerous disadvantages to autografts. There is loss of donor nerve function and the risk of morbidity or neuroma formation at the donor site; caliber mismatch, limited supply, susceptibility to crush injury during harvest, nerve regeneration can be hampered by perigraft scar tissue ingrowth; and outgrowing or regenerating axons may escape to extraneural tissue.

The two major factors that determine nerve regeneration are the rate and quality of axonal outgrowth, and the orientation and specificity in growth of regenerating axons. In addition, the Schwann cells play several key roles in supporting nerve regeneration. A number of trophic factors that support neuronal survival and axonal attachment and growth are synthesized by Schwann cells. The Schwann cells also provide guidance to injured axons and remyelinate these axons.

Other investigators have studied, with some success, alternative approaches to the problem of nerve repair by using nerve guidance channels, including both biological and synthetic nerve guide channels. Examples of the materials used include silicone tubes (Lundborg, et al., 1982, Exp. Neurol. 76:361-375; Williams et al., 1984, Brain Res. 293:201-211), fibronectin mats (Whitworth et al., 1995, J. Hand Surg. 20:429-436), denatured skeletal muscle or muscle basal lamina (Glasby et al., 1986 J. Hand Surg. 11:347-351; Fawcett et al., 1986, J. Neurosurg. 65:354-363), human amniotic membrane (Davis et al., 1987, Brain Res. 430:1-10), veins (Tang et al., 1993, J. Hand Surg. 18:449-453), polyglycolic acid-collagen tubes (Matsumoto et al., 2000, Brain Res. 868:315-328; Kiyotani et al., 1996, Brain Res. 740:66-74; Brown et al., 1996, J. Reconstr. Microsurg. 12:149-152), polylactide scaffolds (Maquet et al., 2000, Peripheral Nerve Regeneration, _{——}:639-651), polyα-hydroxyacid (Oudega et al., 2001, Biomaterials 22:1125-1136), poly-L- lactic acid and polylactic-co-glycolic acid (Hadlock et al., 1998, Arch Otolaryngol Head Neck Surg. 124:1081-1086), polyDL-lactide-ε-caprolactone (Meek et al., 1997, Microsurgery 17:555-561), and acrylonitrile:vinylchloride copolymer (Xu et al., 1995, The Journal of Comparative Neurology 351:145-160). Unfortunately, no method of bridging a nerve defect has proven to be clinically effective.

Yet further investigations are directed to biosynthetic alternatives for nerve repair. (G. K. Frykman, J Hand Ther, vol. 6, pp. 83-8., 1993; T. W. Hudson, et al., Orthop Clin North Am, vol. 31, pp. 485-98., 2000) Biosynthetic conduits have shown to be capable of directing axonal sprouting from the regenerating nerve end and to provide a conduit for diffusion of growth factors secreted by the damaged nerve stumps. (T. W. Hudson, et al., 2000) Current designs shown to support nerve regeneration include the use of biodegradable synthetic materials. (G. R. Evans, et al., Biomaterials, vol. 20, pp. 1109-15., 1999; F. J. Rodriguez, et al., Biomaterials, vol. 20, pp. 1489-500., 1999; 6. S. Yang, et al., Tissue Eng, vol. 7, pp. 679-89., 2001) and direct incorporation of exogenous factors such as extracellular matrix molecules, (R. D. Madison, et al., Brain Res, vol. 447, pp. 325-34., 1988; T. C. Holmes, Trends Biotechnol, vol. 20, pp. 16-21., 2002; J. C. Schense, et al., Nat Biotechnol, vol. 18, pp. 415-9. 2000; R. Biran, et al., J Biomed Mater Res, vol. 55, pp. 1-12., 2001) cell adhesion molecules, (K. Webb, et al., Biomaterials, vol. 22, pp. 1017-28., 2001) growth factors, (J. M. Laird, et al., Neuroscience, vol. 65, pp. 209-16., 1995; G. Terenghi, J Anat, vol. 194, pp. 1-14., 1999) or Schwann cells. (V. Guenard, et al., J Neurosci, vol. 12, pp. 3310-20., 1992; D. H. Kim, et al., J Neurosurg, vol. 80, pp. 254-60., 1994; D. J. Bryan, et al., J Reconstr Microsurg, vol. 12, pp. 439-6., 1996; A. D. Ansselin, et al., Neuropathol Appl Neurobiol, vol. 23, pp. 387-98., 1997) These, and combinations thereof, have been shown to increase the gap length that can be successfully repaired. (K. Matsumoto, et al., Brain Res, vol. 868, pp. 315-28., 2000)

Longitudinally oriented bioabsorbable filaments have been proposed to direct axonal growth, (N. Rangappa, et al., J Biomed Mater Res, vol. 51, pp. 625-34., 2000) and several strategies have been reported to obtain aligned collagen matrices. (N. Dubey, et al., Biomaterials, vol. 22, pp. 1065-75., 2001; P. X. Ma and R. Zhang, J Biomed Mater Res, vol. 56, pp. 469-77., 2001; S. Yoshii and M. Oka, J Biomed Mater Res, vol. 56, pp. 400-5., 2001) Recently, PGA collagen tubes filled with laminin-coated collagen fibers were reported to support regeneration over 80-mm gaps in dogs. (K. Matsumoto, et al., Brain Res, vol. 868, pp. 315-28., 2000) To better resemble the natural microanatomy of peripheral nerves, novel polymer scaffolds have been reported to form organized arrays of open microtubules. (P. X. Ma and R. Zhang, 2001; V. Maquet, et al., J Biomed Mater Res, vol. 52, pp. 639-51., 2000) Alternatively, guidance channels containing longitudinally aligned channels have been constructed. (T. Hadlock, et al., Tissue Eng, vol. 6, pp. 119-27., 2000) However, the random formation of microtubes results in multidirectional axonal growth, in which cell migration occurs primarily along the outer surface of the polymer implant but not within the microtubes. (P. X. Ma and R. Zhang, 2001) In those instances in which Schwann cells are seeded inside the multiluminal conduits, the non-transparent nature of most polymers precludes the verification of cellular fate prior to grafting and during regeneration. Thus, despite the recent progress in the engineering of biosynthetic nerve prosthesis, only modest effects on nerve regeneration and functional recovery have been observed.

Current bridge strategies are based on using neural cells or tissue and extracellular matrix to provide a permissive cellular bridge allowing axons to grow across the lesion site. Nerve grafts containing viable Schwann cells support axonal regeneration more efficiently than nerve grafts composed of basal lamina scaffold only. This biodegradable guidance conduit with a viable cable of Schwann cells can be transplanted into a variety of peripheral or spinal cord transection injuries to increase graft-host integration, and to promote sprouting and regeneration of damaged axons in central nerve system tissue.

There are several limitations for the available biodegradable nerve conduits under current investigation. The manufacture of nerve conduit is rather complicated, takes a long time, and is used with solvent that is toxic to the cells. The dynamic seeding of Schwann cells requires special equipment, involves three stages, and the stage for loading of cells alone can cost several hours. The material for the conduit is not transparent and is not suitable for real time observation and dynamic follow up of cellular and/or tissue morphology and viability. Nerve conduits based on different types of gel or extracellular matrix (ECM), alone or mixed with Schwann cells are also under investigation. However, the current models appear to lack longitudinal straight multiple channels inside the conduit to provide a physically permissive contact guidance structure to direct the nerve regeneration.

Therefore, an improved nerve conduit is needed to provide a unique combination of physically permissive contact guidance structure (multiple channels), favorable biological substrates (ECM) and crucial viable cell components (Schwann cells) for neurite attachment and extension to promote PNS and CNS regeneration.

### SUMMARY OF THE INVENTION

The present disclosure addresses at least a portion of the shortcomings in the prior art by providing a transparent nerve conduit made of a biodegradable polysaccharide such as an agarose gel. The disclosed agarose-based multi-channel conduit allows for the controlled culture and evaluation of cellular elements, normal or genetically-engineered, seeded into longitudinally arranged channels. The transparent nature of this conduit renders it possible for the real time observation and dynamic follow up of cellular viability and morphology prior to nerve grafting.

Multiple longitudinal channels are preferably created within the conduit by using fibers or wires that span the entire length of the conduit and are removed after the gel has formed resulting in multiple channels that are suitable for use as cell growth channels. Additionally, cells may be loaded into the channels by any effective means and may be loaded in such a way that a gradient is created within individual channels. A gradient may be a cell density gradient, or alternatively, a channel may be loaded such that a gradient of secretion products from the cells is created. In preferred embodiments, cells, such as Schwann cells may be coated with extracellular matrix (ECM) and loaded into the channels by capillary force. This conduit provides a physically permissive contact guidance structure (channels), favorable biological substrates (ECM) and crucial cell components (Schwann cells) for neurite attachment and extension along the longitudinal axis.

The preferred nerve conduit as disclosed herein provides greater flexibility for custom fabrication of a cell scaffold designed for a particular nerve to be repaired. The whole procedure to prepare the nerve conduit is relatively easy, requires no special equipment, and can be accomplished within a relatively short period of time as compared to current methods. This disclosure thus enables the rapid production of a nerve growth channel individually tailored to the repair of a particular injury.

When, in certain preferred embodiments, cultured Schwann cells (SCs) are loaded into these channels, the cells are attached, elongated and a three-dimensional viable tissue structure is formed within 2 to 3 hours. The early presence of the interaction of ECM with cellular components (or genetically modified cells secreting nerve growth factors) provides an ideal environment for stimulation of the early phases of axon regeneration. By forming an organized cable, the aligned Schwann cells may serve as a preformed scaffold for elongating axons. The initial nerve regeneration events occur faster, and regeneration is thus accelerated. Providing an early formed Schwann cell cable is contemplated by the inventors to increase the gap distance that can be bridged and to decrease the reconnection time with a target tissue, thus improving functional recovery. Therefore, the ability to use the disclosed nerve conduit with SCs and ECM to form a highly organized, longitudinal, growth-permissive framework at such an early stage is of substantial importance to support direct axonal regeneration across a lesion site and to restore normal neuron function.

The present disclosure is not limited to regeneration of nerve cell connections or to nerve tissue of either the central or peripheral nerve systems. For example, a similar strategy may be applied to the engineering of other types of tissues such as muscle, tendon, blood vessel, or organs such as pancreas or liver. The disclosed scaffolds offer particular advantage in that the cells may be loaded rapidly in a controlled manner and the growth of such cells can be visually monitored both for cell growth and for response to various growth factors or chemical or biological agents.

A further advantage of the present disclosure, therefore, is the transparent nature of the aragose scaffold. The cell scaffold is thus useful as a visible 3-dimensional tissue culture system in which cells can be directly observed under a variety of conditions. Cellular behavior can thus be determined (in vitro and in vivo) in the presence of various chemical or biological agents, pharmaceuticals, nutrients, toxins, metabolites, metals, or in the presence of various cell types to monitor cell-cell interactions, cell motility, viability, binding, aggregation, tumor growth, or cell death, as influenced by both their intrinsic genetic programs and their extracellular environments.

The cell scaffolds disclosed herein thus may serve as a unique nerve conduit and are contemplated to find widespread use for clinical nerve repair of damaged peripheral nerve, spinal cord and central nerve systems. The cell scaffolds of the present disclosure can also be used in tissue engineering of other types of tissues, in cell biology studies and pharmaceutical studies.

The present invention may be described, therefore, in certain embodiments as a transparent agarose cell scaffold comprising a plurality of channels contained in the scaffold, wherein the scaffold is configured such that biological cells loaded into the channels are observable by microscopy during growth of the cells in the channels. Scaffolds may be substantially cylindrical, rectangular or square in shape with the channels extending the entire length of the scaffold body. The channels are thus effective to provide a liquid conduit from one end of the scaffold to the other end of the scaffold. This arrangement allows the loading of cells quickly in the channels by capillary action and allows the loading of cells in a gradient from one end of the channel to the other.

A preferred scaffold is one that is loaded with cells within the channels and particularly with biological cells, which are defined as the cells of a biological organism or even a microbiological cell culture, and preferably with eukaryotic cells such as plant, yeast or animal cells, preferably mammalian cells and more preferably human cells. In certain embodiments the cells are glial cells, endothelial cells, muscle cells, or neurons.

It is an aspect of the present invention that the cells may be loaded into the channels to form a gradient along at least a portion of the channels. The gradient may be formed by loading a higher cell density at one end and a lower cell density at the opposing end of a channel, or by loading two or more cell populations that differentially express a gene product of interest such as a growth hormone or a receptor, for example. The cells may be loaded by capillary action by contacting a cell culture with one end of the channel while contacting the opposing end with an absorbent media such as an absorbent paper. Channels may also be loaded by microsyringe or any method known in the art.

It is a further aspect of the invention that only one type of cell may be loaded into the channels for certain purposes, or that two or more cell types may be loaded. When two or more cell types are used, individual channels may contain only a single type of cell or a mixture of cell types as required for any particular purpose. For example, a first channel may be loaded with a first single type of cell and a second channel may be loaded with a second and different single type of cell, or two or more cell types may be loaded into a single channel. In preferred embodiments directed at studies of animal or human cell responses, preferred cells would include, but are not limited to Schwann cells, myoblasts, 3T3 cells, PC 12 cells, NG108 cells, astrocytes, oligodendrocytes, fibroblasts, hepatocytes, chondriocytes, osteoblasts, or stem cells.

The cells that are loaded in the channels are, in certain embodiments, cells that naturally or recombinantly express one or more cell growth factors, and in preferred embodiments, the cells express a gradient of nerve growth factors to serve as a conduit for directed axon growth. For example, in certain embodiments the cells may express nerve growth factor (NGF), ciliary neurotrophic factor (CNTF), glial cell line derived neurotrophic factor (GDNF), brain-derived-neurotrophic factor (BDNF), neurotrophin-4/5 (NT-4/5), neurotrophin-3 (NT-3), insulin-like growth factor (IGF-I), basic fibroblast growth factor (FGF-2), leukemia inhibitory factor (LIF), transforming growth factors (TGFs)-β, GGF, or any combination of these. In certain embodiments the cells may express one or more cell adhesion molecules, such as L1, N-CAM, NgCAM/L1, L1 L2/HNK-1 or N-cadherin, for example, or they may express one or more extracellular matrix molecules, such as laminin (LN), heparan sulphate proteoglycans (HSP), tenascin, or fibronectin (FN), or one or more chemoattractants, such as Slit, Netrin, or Ephrins, for example.

As was stated above, the cells may express growth factors, cell adhesion molecules, extracellular matrix molecules, or chemoattractants either naturally or as a result of the genetic engineering of the appropriate gene into the cells for expression. In certain embodiments, all the cells to be loaded will express the desired molecules at substantially the same level, and in those cases, the cells may be loaded to create a cell density gradient in the channel. In some embodiments, the cells may differentially express the preferred molecule due to factors known in the art such as a difference in copy number of recombinant gene, or by the use of promoters or enhancers that direct different levels of expression. For example, a recombinant gene may be expressed under the control of an inducible promoter that responds to an external or internal signal to initiate transcription of the gene and the level of expression may thus be controlled within the channels of the described cell scaffolds. The differential expression of recombinant genes under various conditions does not, in and of itself, constitute the present invention, and any method known in the art would fall within the scope of the present disclosure.

In certain aspects, the present invention may be described as a method of constructing a cell growth scaffold, the method including forming a substantially cylindrical agarose body including an outer diameter and two ends, and further including multiple channels within the interior of the body extending from the first end to the second end, wherein the channels provide a fluid connection through the body from the first end to the second end of the body, and further wherein the body is substantially transparent such that cells within the channels are visible to microscopy techniques during use. The cells may be visually detected either by staining techniques known in the art, or more preferably by fluorescent labels or tags that are bound to the cells or even genetically expressed by the cells to be used. In certain embodiments, green fluorescent protein (GFP), or a red (DiI) or a green (DiO) lipophilic dye may be used.

The cell scaffolds of the present disclosure are preferably made of linear polymers that form a gel at room temperature that is held together without cross-links by hydrogen bonds. More preferred for use in constructing the cell scaffolds is a neutral polysaccharide derived from agar that may be a fraction of agar generally including galactose and altered anhydrogalactose residues, better known as agarose, and more preferably of ultrapure™ agarose, commercially available from Gibco BRL. The concentration of the agarose will vary depending on the particular use, with higher concentrations resulting in a denser, more resilient body. The concentration of agarose generally falls within the range of from about 1% to about 1.5%, or from about 0.5% up to about 3%, or from about 4% to about 5% for certain uses. The scaffold is made by standard techniques in which a tube, cylinder, or wax structure is used as a mold and filled with melted agarose. The agarose is allowed to gel at room temperature or below as needed. The agarose is preferably dissolved in a benign salt solution such as phosphate buffered saline (PBS). The preferred size of the scaffold for use as a nerve regeneration conduit is such that the body has an outside diameter of about 3 mm and an inner diameter of about 2 mm, and where channels are formed in the body of the scaffold by a fiber or wire that has diameter of from about 0.06 mm to about 0.17 mm. The body will typically include from about 10 to about 20 channels, but more or less may be used as needed.

Certain aspects of the invention may also be described as methods for screening a candidate substance for an effect on the growth, motility or viability of a target cell. The described methods may include providing a cell scaffold as described above in which one or more channels are loaded with target cells; contacting the target cells in the channels with the candidate substance; observing the contacted cells microscopically; and comparing the contacted cells to identical control cells in the absence of the candidate substance; wherein a difference in the growth, motility or viability of the contacted cells relative to the identical control cells is indicative of an agent that affects the growth, motility or viability of the target cells.

In preferred methods, the target cells may include Schwann cells, myoblasts, 3T3 cells, PC 12 cells or stem cells, and the cells or the channels may be coated with an extra cellular matrix such as about 10% ECM gel, or the cells may be coated with the matrix, such as Schawnn cells coated with ECM gel, for example. In certain embodiments, the agarose gel may include pores, permitting nutrients and gas to pass through the pore of the conduit but inhibiting neurites to grow through the pores. Preferred gels remain transparent for at least three weeks in vitro and the cell morphology may thus be observed under microscopic examination for any given point of real time observation or for regular follow up study of cellular morphology changes and genetic and/or metabolic function in a three-dimensional environment.

The inventors have shown, by the use of the described methods, that Schwann cells may be loaded into the channels within one minute by capillary action, and that within two hours after cultured Schwann cells are loaded into the channels, the cells are attached, elongated and a three-dimensional longitudinal viable tissue structure is formed inside the channels. The present methods are thus an ideal method for the growth of a scaffold for early nerve regeneration. The described scaffolds are also useful for the growth of different types of cells for use in the tissue engineering of muscle, tendon, blood vessel, pancreas or liver. For example, the inventors have demonstrated that myoblasts loaded into the channels adhere to the interior surface of the channels and grow within 24 hours. In preferred embodiments the myoblasts express green fluorescent protein (GFP), and more preferably the myoblasts are transfected with a virus that expresses the GFP in the myoblasts. The described culture of myoblasts thus may serve as a model for gene therapy or as a model for local drug delivery.

The present inventors have also shown that 3T3 cells may be loaded into the channels, where they adhere to the interior surface of the channels and grow for several weeks. The 3T3 cells grow in single cell, fibroblast-like and cluster-like growth patterns. This method of culture of 3T3 cells allows the direct visualization of the 3T3 cell growth pattern, and is a useful tool in testing the efficacy of anti-cancer agents, and to compare the genetic and cellular differences between 2D and 3D cell growth of the cells.

In further embodiments, PC 12 cells loaded into the channels, show neurite formation, and are an effective model for an in vitro electrophysiology study of cellular function, and also serve as a model for screening the effectiveness of pharmaceutical agents such as antiepileptic agents, for example.

In still further embodiments, two or more types of cells may be loaded into single channels for the study of cell/cell interactions, as in the examples wherein myoblasts are loaded with Schwann cells, or even wherein Schwann cells are loaded with PC 12 cells, effective to investigate the cellular interaction or communication among different type of cells.

As was described above, the cells may be loaded to form a gradient of cells, or two or more populations of cells may be inducible for differential expression of one or more gene products. In certain embodiments, stem cells may be loaded into the channels, and would serve as a universal conduit for tissue engineering of tissues or organs.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A is a graphic representation of a method of microchannel casting into an agarose conduit in which (a) fibers are placed in a cylindrical tube, (b) the tube is filled with melted agarose which is allowed to gel at room temperature or below, (c) the fibers are removed to create cell growth channels, and (d) the channels are seeded with cells.

FIGS. 1B and 1C are photomicrographs of a conduit. A conduit is shown with a fiber in place in FIG. 1B. Bar=1 mm in FIG. 1B and 0.5 mm in FIG. 1C.

FIGS. 2A-d demonstrate a cellular density gradient in a single channel of a transparent conduit. The cellular gradient was formed by regulated the capillary force during seeding. The photographs are of 3T3 fibroblasts 2 ours after loading show low (A, B) medium (C) and high (D) cell titers along the channel. Bar=125 μm.

FIG. 3A 3T3 fibroblast growing in a linear shape at low titer in the presence of extra cellular matrix. Bar=125 μm

FIG. 3B 3T3 fibroblasts growing in tubular shape in the channel at high titer in the presence of extra cellular matrix. Bar=250 μm

FIG. 3C Low titer 3T3 cells loaded in absence of extracellular matrix gel, grew as a cluster. Bar=250 μm

FIG. 3D As indicated by DAPI nuclear staining, the clusters shown in FIG. 3C proliferated in culture for more than 3 weeks, progressively filling the lumen of the channels without disrupting their shape. Bar=250 μm

FIGS. 4A-D Time course of Schwann cell growth inside the multichannel conduit. Schwann cells displaying their characteristic fusiform shape were cultured at high purity (A). Dissociated cells from these cultures were photographed immediately (B) and 2 hrs (CD) after seeding. Two hours after seeding fusiform Schwann cells were observed to elongate inside the channel (D) forming a long cable-like structure (C). Bar=2 mm (A), 0.5 mm (B), 125 μm (D).

FIG. 5A. PC-12 cells were successfully differentiated in the presence of NGF and their neurites were observed grow along the luminal surface of the channels, respecting their borders.

FIG. 5B Muscle cells spread and grew indistinguishable from their 2D sister cultures.

FIG. 5C Schwann cells were further identified by their expression of the glial filament S-100.

FIG. 5D Schwann cells were also further identified by their expression of primary DRG sensory neurons immunolabeled with an anti-peripherin antibody. Neurite extension was restricted to the lumen of the channels as indicated by the apparent turn of neurites at the border of the channel (FIG. 5A, and arrows in FIG. 5D). All photographs were taken at 24 hrs after seeding. Bar=50 μm.

FIGS. 6A-D. Gene-transfer of sensory neurons cultured inside the channels. Color combined photomicrographs of GFP expression (green) by DRG explants placed inside individual channels, and the conduit visualized by phase contrast and pseudocolored in blue to enhance the visibility (A,B). After 4 days in culture GFP-positive DRG neurons were observed to growth neurites within the explants and stop at the explant-channel interface (C, D). Two days later some of these neurons were observed to extend their axons along the uncoated agarose channel (arrows indicate GFP-positive axons). Bar=250 μm.

FIGS. 7A-D. Directed and enhanced axonal growth in transparent conduits pre-seeded with Schwann cells. Double immunofluorescence using specific neuronal (peripherin-positive: green) and Shwann cell (S-100 positive: blue) antibodies were use to visualize these cells, 24 h after DRG seeding into a Schwann cell containing conduit. As indicated by the arrows, DRGs showed enhanced neurite growth reaching 1 mm in one day. Although the general direction of neurite growth is dictated by the channels (arrows in A), the Schwann cells inside the lumen (arrowheads in B-D) can clearly deviate the trajectory of ingrowing axons (arrows) at the point of intersection (asterisk). Bar=50 μm.

FIGS. 8A-C illustrate an embodiment of production of a wax mold to produce transparent multichannel matrices.

FIGS. 9A-C illustrate an embodiment of production of a wax mold to produce multiple transparent multichannel matrices.

FIG. 10 is photographs of DiI/DiO labeled Huvec cells seeded into alternating chambers of a transparent multichannel matrix. The left column is at 5×magnification and the right column is at 20×magnification. DiI labeled cells appear green and DiO labeled cells appear red. The bottom row illustrates segregated cell types in separate adjacent channels.

FIG. 11 is photographs of Dil/DiO labeled Huvec cells seeded into a single channel but separately distributed.

FIG. 12 is a photograph of an example of a device configured to provide separate media to individual channels of a transparent multichannel matrix.

### DETAILED DESCRIPTION OF THE PREFERRED EMBODIMENTS

In certain embodiments, the present disclosure provides a scaffold for cell growth in which the cells grow in channels through the scaffold, and in which the scaffold is transparent such that the cell growth and morphology can be directly observed. The scaffolds may also be described in certain embodiments as a polysaccharide gel guidance conduit that preferably includes, or the body of which is primarily composed of agarose. This scaffold may be used for the growth and study of any type of biological cells including eukaryotic cells and prokaryotic cells, including cells of animal, plant or yeast origin, or single cell microorganisms. The use of the disclosed scaffolds would include the growth of cells for tissue engineering applications including, but not limited to growth of organ tissue such as liver or pancreas tissue, the growth of skin, muscle, connective tissue, vascular tissue, or nerve cell elongation for nerve regeneration. In addition, cells grown within the scaffold may be directly observed microscopically, and also may be labeled for identification of particular cell types within a mixed culture of cells within an individual cell growth channel, or within the scaffold as a whole. This advantage provides the ability to observe cell growth morphology under a variety of controllable conditions of media, environmental factors or the presence of hormones, growth factors, nutrients, metabolites, pharmaceutical agents or small molecules. A particular advantage is offered by the ability to observe tumor cells in a three dimensional morphology and the response of such cells to chemotherapeutic agents. Another advantage is offered by the ability to grow mixed cultures of cells, either in individual channels, or mixed within channels in order to directly observe cell to cell interactions and for the engineering of complex tissues.

In certain embodiments, the use of the disclosed cell growth scaffolds includes methods to promote Schwann cell attachment, elongation and formation of a longitudinal viable cable structure within channels within the conduit for early nerve regeneration, and methods for allowing real time observation or dynamic follow up of cellular morphology changes and genetic and/or metabolic function in a three-dimensional environment. The conduits or scaffolds of the present disclosure provide further advantages in that the biodegradable material has great flexibility to allow custom fabrication of conduits for a particular nerve to be repaired. For example, Schwann cells may be isolated from a nerve injury patient, expanded and then cultured in a nerve growth conduit of the present invention to quickly provide a scaffold for nerve regeneration. The whole procedure to prepare the nerve conduit is simple and can be accomplished within a short time period. This is especially important in nerve regeneration since the size and dimensions of the conduit and channels for repairing a damaged nerve can sometimes only be decided during the surgical procedure.

In preferred embodiments, the conduits are made of clear and transparent biodegradable material without the use of materials that are toxic to the cells. This transparent media allows real time observation and dynamic follow up of the cellular and/or tissue morphology during growth. Therefore, the devices described herein are useful as experimental tools by providing a three dimensional tissue culture system, available for investigating the behavior of a variety of cells influenced by both their intrinsic genetic programs and their extracellular environments, both in vitro and in vivo.

The adhesion extra-cellular matrix (ECM) inside the channels supports myelin-producing Schwann cells (SCs), that are important to axon initiation, elongation, branching and long term survival and differentiation. Schwann cell cultures may be established, for example, as described by Ansselin et al. (In Vitro Cell Dev. Biol. 31:253, 1995) or by any culture technique known in the art. The conduits of the present disclosure also provide the important benefit of formed Schwann cell scaffolds that can be used early in the process of the repair of peripheral nerve injuries, spinal cord injuries or central nerve system damage. By providing formed Schwann cell scaffolds as soon as possible after nerve injury, the cells inside the channels not only act as mechanical cues to increase graft-host integration, but they also promote sprouting and regeneration of damaged axons, and may bypass the initial nerve regeneration events normally associated with nerve injury. In this way, the conduits are contemplated to accelerate regeneration, increase the gap distance that can be bridged, and decrease reconnection time with target tissue, thus improving functional recovery.

Thus, in one aspect, the present disclosure provides biosynthetic prostheses as an alternative to peripheral nerve autograft in clinical nerve repair. To this end, ex vivo gene therapy may be used to induce expression of relevant cell adhesion molecules (CAMs) and nerve trophic factors (GFs) in either autologous or allogenous Schwann cells prior to their incorporation into biodegradable nerve guides. This approach provides trophic support to injured neurons for enhanced survival, an adhesive substrate for suitable axonal regrowth, and a gradient of neurotrophic factors to promote and direct axonal regeneration, using an implantable matrix made of both synthetic and cellular components. A gradient of neurotrophic factors may be provided by various techniques, including loading the cells into the channel in a density gradient, or by controlling gene expression levels in the cells to create a gradient of the growth factors.

A typical nerve growth scaffold connects a severed nerve in which a proximal nerve stump and a distal nerve stump are placed adjacent respective ends of the scaffold. The scaffold is composed of a transparent gel matrix and contains multiple pre-cast mini-channels. During use, the conduit is preferably enclosed in an external biodegradable connecting tube. During use, the channels are loaded with Schwann cells in a gradient to promote nerve cell growth through the channels from the proximal to the distal stump of the severed nerve.

Nerve Regeneration

After injury, axonal regeneration proceeds spontaneously in the peripheral nervous system with some degree of tissue and topographic specificity, if nerve continuity is either preserved or reestablished. The healing of nerve injuries takes place in an environment of intense cellular proliferation (i.e., fibroblasts, endothelial cells, Schwann cells). Schwann cells, the myelinating or ensheathing glial cells of the peripheral nervous system, proliferate and align, forming bands through which regenerating axons grow. Activated Schwann cells also upregulate the synthesis of cell adhesion molecules (CAMs) and neurotrophins that, in turn, influence the growth and orientation of regenerating axons. Cell adhesion molecules, such as NILE, N-CAM and N-cadherin, provide the substrate that allows axonal regeneration whereas, neurotrophins; such as brain-derived-neurotrophic factor (BDNF), ciliary neurotrophic factor (CNTF), insulin-like growth factor (IGF-I), basic fibroblast growth factor (bFGF), and nerve growth factor (NGF), increase neuron survival and promote axonal outgrowth. In the present disclosure, the use of a gradient of Schwann cells, or the use of a genetic gradient, in which nerve growth factors are expressed at higher levels in a portion of the population of cells, will create a growth cone of nerve growth factors to direct the axon growth to the distal stump of a severed nerve.

Gene Therapy for CAMS and Neurotrophin Expression

Numerous studies have demonstrated that neurotrophic factors have the potential to enhance neuronal survival and axonal regeneration. However, local and continuous delivery of these molecules is challenging. Gene therapy provides an attractive alternative for delivery of CAMS and neurotrophins in the nervous system by allowing constitutive or inducible induction of gene expression in situ. Manipulation of gene expression in the nervous system has a broad therapeutic value, including enhancement of neuron survival, growth of neuronal connections and increasing the overall regenerative potential of neurons after injury. A part of the present disclosure is the application of adenoviral-mediated gene transfer technology for use in axonal regeneration and functional recovery after spinal cord injury through overexpression of CAMs and neurotrophins. It is understood that alternative methods and vectors would also be useful in the practice of the present invention. For example, other gene transfer vectors that are contemplated for use in the present invention include, but are not limited to vectors derived from retrovirus, lentivirus, herpes virus, and other known mammalian viral vecotors, and also include the use of naked DNA, liposomes, especially cationic liposomes for DNA delivery, ballistic gene transfer, and electroporation, for example. The genes may be under the control of their endogenous promoters, or may be under the control of, or operatively linked to externally regulated promoters such as Tet-on, or estrogen sensitive promoter, for example, or endogenously regulatable promoters, such as a glucose induced promoter, for example. In addition, cell-specific promoters such as the P0 promoter for gene expression in Schwann cells may be used.

In certain embodiments the cells are transformed with recombinant plasmid expression vectors (e.g., Ti plasmid) containing desired gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In mammalian cells, a number of viral-based expression systems may be utilized. In cases where an adenovirus is used as an expression vector, the desired gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts. (e.g., See Logan and Shenk, 1984, Proc. Natl. Acad. Sci. USA 81, 3655-3659). Specific initiation signals may also be required for efficient translation of inserted gene product coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner, et al., 1987, Methods in Enzymol. 153, 516-544).

In certain preferred embodiments an adenoviral vector is used. Replication-defective recombinant adenoviruses may be constructed by the method described by Bett et al., (Proc. Natl. Acad. Sci USA 91:8802, 1994, incorporated herein by reference). The coding region of the gene to be expressed is inserted into a modified pXCJL vector (Romero and Smith, Gene Therapy 5:1612, 1998, incorporated herein by reference) that contains a Rous sarcoma virus long terminal repeat promoter and a bovine growth hormone polyadenlyation signal (pXCRSV) (Smith et al., J. Neurophysiol. 77:3115, 1997). Viral particles lacking the E1 and E3 regions are obtained by cotransfecting equimolar concentrations of the pXCRSV plasmid and the pBHG11 adenoviral genomic vector (Microbix, Toronto, Canada) into 293 cells using a liposomal delivery system (DOTAP, Boehringer Mannheim, Indianapolis, Ind.). Adenovirus containing the proper inserts are plaque purified, propagated in 293 cells, and purified twice in a cesium chloride gradient. Lack of wild type contamination may be confirmed by PCR with primers complementary to the EIA region (Zhang et al., Biotechniques 18:444, 1995). The physical number of viral particles is determined by optical absorbance and the number of infectious particles estimated by counting crystal violet-stained plaques using the agarose overlay method.

Temperature sensitive adenovirus may also be used in the present invention. Temperature sensitive adenoviruses are generated by subcloning the ts mutation from HSts125 (Ensinger and Ginsberg J. Virol. 10:328, 1972; Kruijer et al., Virology 128:140, 1983) into the pBHG11 plasmid. The HSts mutation includes a point mutation that converts a proline into a serine at position 413 of the E2A coding region that renders the adenovirus unable to replicate or to synthesize late viral proteins when grown at 39.5° C. A 6710 bp fragment containing the ts mutation may be excised from the HSts125 viral genome using the SfiI restriction enzyme and ligated into the SfiI-restricted pBHG11 vector.

The adenoviral vector may then be used to infect the cell population to be loaded into a cell growth conduit. The number of viral particles per cell may be varied for different cell populations to create an expression gradient of an effector molecule such as a nerve growth factor, for example, within a conduit of the present invention.

Cell Growth Assays

An aspect of the present disclosure is the use of a transparent conduit in assays of the effects of various agents on cellular growth, motility, and viability, for example. In a preferred embodiment, the conduit may be used to test the growth of neurons in response to various cell adhesion molecules such as NILE and nerve growth factors such as bFGF, NFG, and BDNF, expressed from engineered, or recombinant Schwann cells. The disclosed conduits may be used for example, to examine axonal growth of both motor and sensory neurons on genetically-transformed Schwann cells preseeded into the conduits. By use of the assays, one can determine if overexpression of NILE alone or in combination with the growth factors improves axonal growth over that achieved on acellular fibers. The effect of gradients of growth factors and cell adhesion molecules may also be determined.

Material and Methods:

Preparation of the Cylindrical Agarose Matrix with Pre-Cast Microchannels

Ultrapure agarose (1%-1.5%: Gibco BRL) is dissolved in phosphate buffer saline (10 mM, pH 7.4, Sigma) at 56° C. and injected into a plastic tube that contains multiple fibers or wires inside. The tube has an outside diameter of 3 mm, an inner diameter of 2 mm, and a longitude or length of approximately 10 mm. The plastic fibers inside the tube are of approximate 0.06-0.17 mm in diameter, 10 mm in length, and 10-20 in number. After polymerization of the gel at room temperature or 4° C. for 15 minutes, the multiple channels within the conduit are created by carefully pulling the fibers from the gel.

Loading of the Cells Into the Conduit

Different types of cells, such as glial, endothelial cells, muscle cells, and/or neurons are suspended in a 10% cellular matrix gel (Matrigel) to increase cell attachment within the channels. The cells are then loaded into the micro channels by capillary force. One end of the multichannel conduit is placed in contact with the cell suspension solution and a piece of filter paper is placed at the other end of the conduit. By this method, the cells are loaded inside the channels in approximately 1 minute. Cell numbers in the cell suspension can be varied to achieve the desired cell density within the channels. Alternatively, the cells may also be loaded into the channels using multi-barrel syringes or glass micropipettes. The last method can be more time consuming but allows for more control in loading the cells.

Creation of a Cellular and/or Molecular Gradient

Creation of cellular gradients within the channels is achieved by regulating the capillary force applied to the channels at the time of loading the cells. Thus, by reducing the time of application or the capillary force itself, the number of cells reaching the other end of the tube will be gradually reduced compared to those that are loaded in the segment of the conduit in direct contact with the cell suspension. If a micropipette is used, then the transparency of the conduit allows for the visual delivery of cells within the channels in a gradient density.

A molecular gradient can be engineered within the tube by at least two different methods. One is the creation of the cellular gradient as previously described, using cells that are modified genetically to express a particular gene. Because the cells are loaded in a gradient, gene expression within the channel is expected to form a gradient. A second method is that in which the cells are loaded at constant density inside the channels, but gene expression of those cells forms a gradient per se. This is achieved by gradually varying the number of gene copies per cell, or gene-transfection units per cell within the channels. In certain embodiments, a plurality of cell populations may be loaded sequentially into a channel to create such a gradient.

The genes may be transferred into the cells by methods known in the art, such as by transfer of naked DNA, liposomes, ballistic gene transfer, and electroporation, etc. Other methods would include the use of vectors such as adenoviral, lentivirus, herpes virus, vectors, etc. The gene expression may be under the control of externally regulatable promoters, such as the Tet-on, or estrogen induced expression promoters, or of endogenously regulatable promoters such as glucose induced expression, for example. The use of cell specific promoters is also contemplated, such as the use of the P0 promoter for gene expression in Schwann cells.

In addition, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid iphosphatase, and the promoters of the yeast alpha-mating factors.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

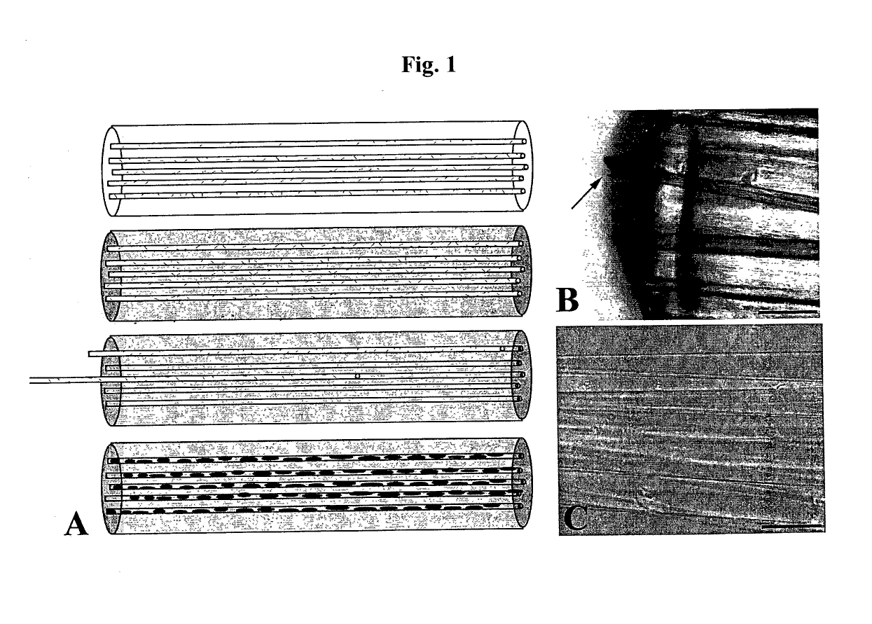

The method of casting microchannels into an agarose matrix (transparent multi-channel conduit, TMCC) is illustrated in FIG. 1. Multiple plastic fibers (0.06×0.17 mm in diameter) were placed inside a plastic tube (3 mm OD, 2 mm ID, and 10 mm length; FIG. 1A). Ultrapure agarose (1%; Gibco, BRL), a natural polysaccharide consisting of 1,4-linked 3,6-anhydro-α-L-galactose and 1,3-linked β-D-galactose, was melted in pre-warmed (75° C.) PBS, injected into the fiber-containing tube and allowed to gel at 4° C. for 15 minutes (FIG. 1B). The agarose concentration was based on previous reports on the physical properties of this polysaccharide in relation to neurite extension. (T. Hadlock, C. Sundback, D. Hunter, M. Cheney, and J. P. Vacanti, Tissue Eng, vol. 6, pp. 119-27., 2000) The multiple channels within the conduit were created by carefully removing the fibers from the gel (FIG. 1C,), prior to cellular seeding (FIG. 1D).

Cell Culture and Seeding of the TMCC

The conduits were then seeded with several types of cells. Schwann cells were obtained from murine sciatic nerves and cultured in DMEM/10% FBS, PC-12 cells (ATCC CRL-1721) were cultured in RPMI 1640/10% HS/5% FBS, and differentiated by adding 50 ng/ml NGF (Amgen). Myocytes obtained from skeletal muscle, and 3T3 fibroblasts were grown in DMEM/10% FBS. Each of the cells tested were seeded into the channels by placing a sterile cotton tip at one end of the TMCC (capillarity).. The cellular density inside the channels was varied through the use of different cell titers at the time of seeding. Cellular gradients were formed by regulating the capillary force applied at the time of seeding. In order to enhance the ability of the TMMC to allow for cellular attachment and growth, some of the cells were mixed with 10% matrigel (Sigma), prior to seeding.

Immunocytochemistry

Schwann cells in culture dishes were fixed with 4% paraformaldehyde for 20 minutes and permeabilized with 0.2 Triton X-100 for 30 minutes. After blocking with 2% normal goat serum, the cells were incubated with anti S-100 monoclonal antibody (1:500: Sigma) or P 75 polyclonal antibody (1:200:Sigma) overnight at 4° C. Primary sensory neurons were differentiated from Schwann cells by simultaneous incubation of the cell-containing conduits in a mix of anti-100 antibody and an anti-peripherin polyclonal antibody (1:500: Chemicon). Visualization of the primary antibodies was achieved by incubating the tissue with Cy2- and Cy3-labeled secondary antibodies, for 1 hour at room temperature. The cultures were then mounted onto slides, coverslipped and observed using an epifluoresence microscope.

Adenoviral Transformation of Dorsal Root Ganglia Explants

DRG were obtained from adult mice, minced, and placed within the channels. At the time of seeding, 1×10⁷ pfu of an adenovirus encoding for the enhanced green fluorescent protein (GFP) was added to the culture as previously described. (M. I. Romero, N. Rangappa, L. Li, E. Lightfoot, M. G. Garry, and G. M. Smith, J Neurosci, vol. 20, pp. 4435-45., 2000)

Cellular Behavior within the TMCC

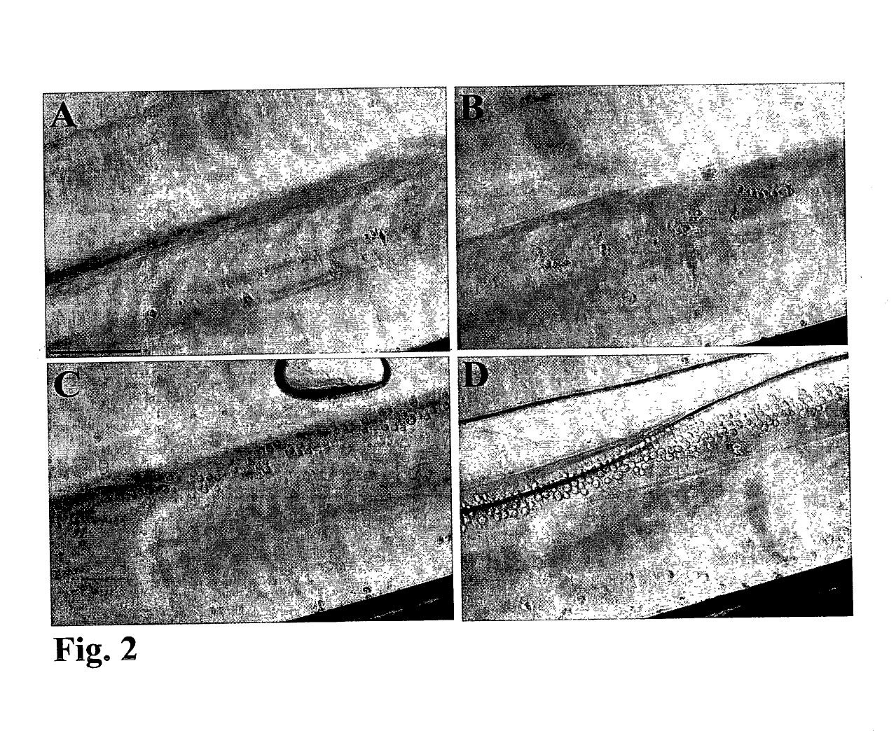

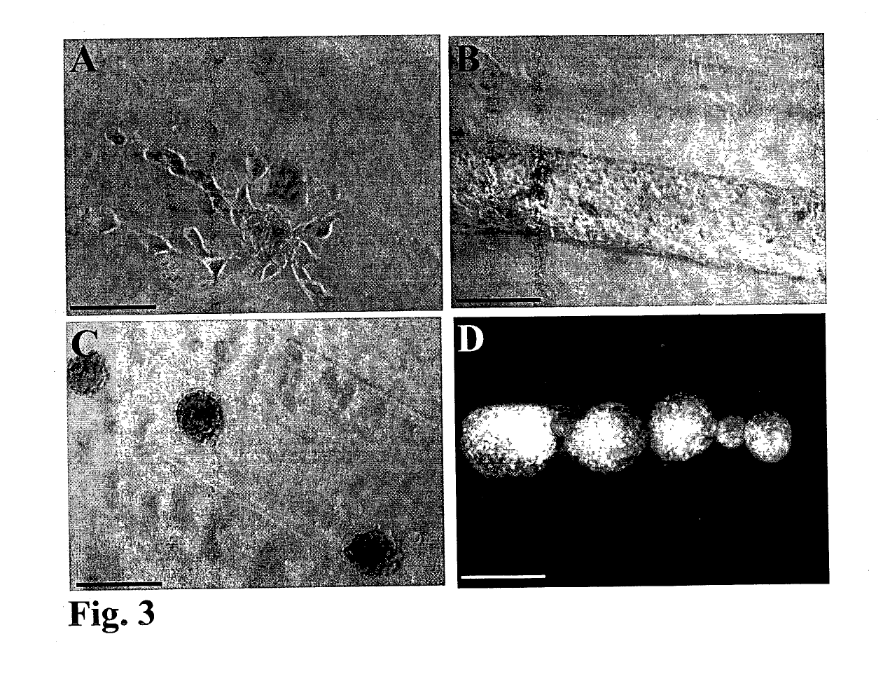

The casting of multiple channels within an agarose matrix was achieved within minutes, in a simple and reproducible manner. The cellular density loaded into the channels of the transparent multichannel conduit (TMCC) was controlled by modifying the capillary force applied at the time of seeding. Utilizing this control, cellular gradients were generated inside individual channels. As demonstrated in FIGS. 2A-D, there is a progressively higher cell titer as one moves from one end (FIG. 2A) to the opposite end (FIG. 2D) of a single channel. Within the TMCC, different cells displayed varied responses depending of both the particular cell type, as well as whether or not the cells were premixed in ECM gel prior to seeding. At low densities (1 ×10⁵ cells/ml) 3T3 fibroblasts were able to attach to the non-coated channels, proliferate, and spread normally within hours after seeding (FIG. 3A). When loaded into the TMCC at higher titers (1×10⁷ cells/ml), these cells were able to form a tubular structure that effectively coated the inside of the channels (FIG. 3B). In contrast, Schwann cells seeded at low densities failed to display normal morphology inside the channels, and in turn grew as microspheres (FIG. 3C). These cells remained viable inside the TMCC for up to 21 days in culture, the maximum observation time in this study, with continuous proliferation, as indicated by the growth and eventually fusion of some of the cellular clusters, as well as the incorporation of the nuclear marker DAPI (FIG. 3D). During the entire culture period the structural integrity of the gel and the channels was maintained, and the cellular growth of these clusters was molded into solid tubular structures, (FIG. 3D).

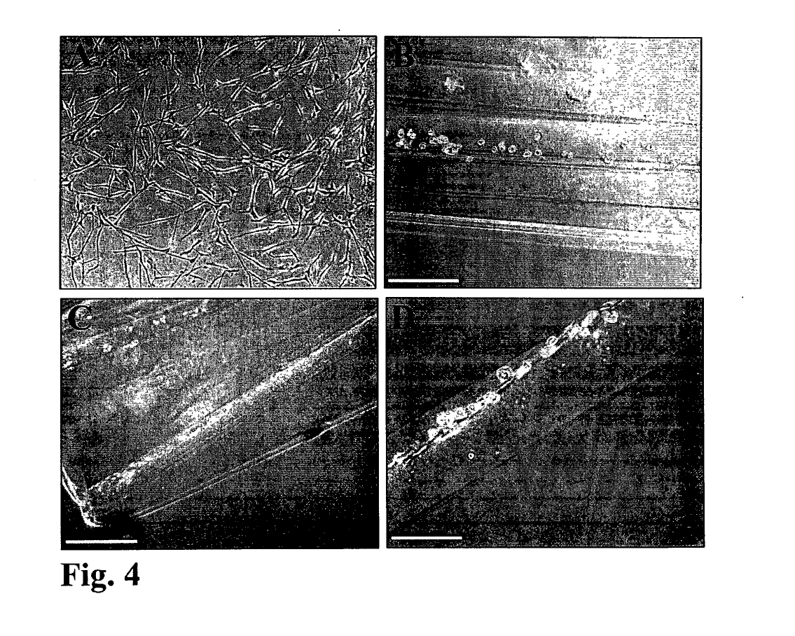

Coating the lumen of the TMCC, and/or mixing the cells with extracellular matrix gel prior to seeding provided a better substrate to all cell types tested, both for attachment and growth. This was clearly demonstrated when Schwann cells (FIG. 4A), were dissociated and mixed with EMC gel prior to seeding. Within 48 hrs after loading, the Schwann cells that initially appeared rounded and discontinuous (FIG. 4B), attached, elongated, and were able to form a longitudinal cellular structure throughout the length of the TMCC (FIG. 4C). At higher magnification it was possible to corroborate the characteristic fusiform shape of Schwann cells, comparable to that of sister cultures (compare FIGS. 4A and 4D).

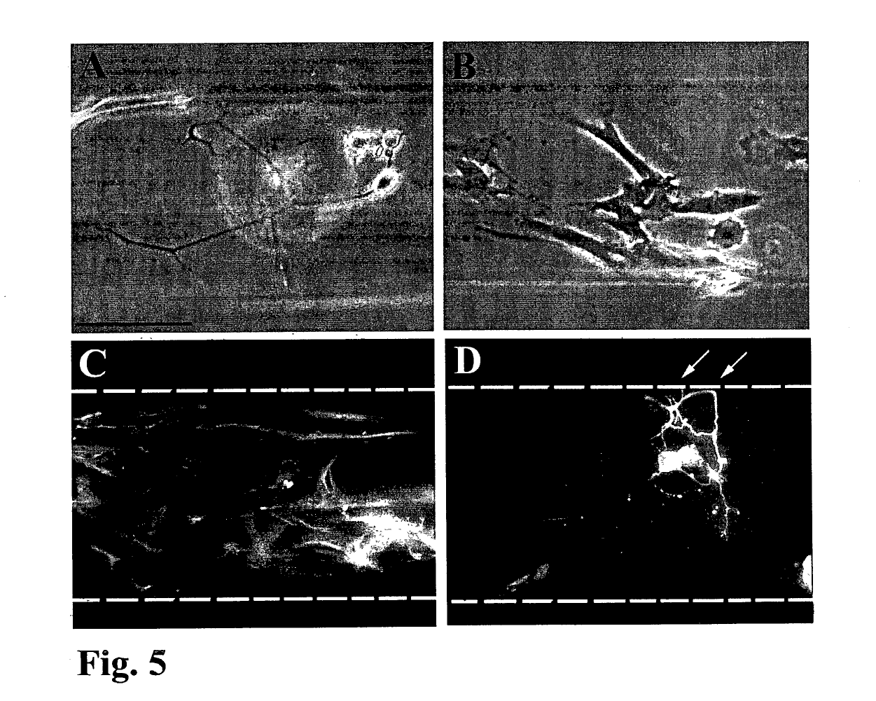

In order to evaluate the ability of the TMCC to support the growth of other cell types, PC 12 cells, muscle cells, and primary sensory neurons from the dorsal root ganglia were cultured inside these conduits. PC 12 cells differentiated into neuronal phenotypes and grew neurites along the lumen of the channels (FIG. 5A). Similarly, muscle cells displayed morphologies that were indistinguishable from that of sister 2D cultures (FIG. 5B). To further corroborate the identity of some of these cells, some of these cultures were stained with specific Schwann cell markers such as S-100 (FIG. 5C) or the low affinity neurotrophin receptor P75. The TMCC was also optimal for the growth of primary sensory neurons. Dissociated adult and neonatal dorsal root ganglion neurons were successfully cultured inside the channels, displaying directed growth. FIG. 5D illustrates a DRG neuron immunostained with an anti-peripherin antibody. The ability of these neurons to extend neurites inside the channels is clearly demonstrated. As shown, the agarose effectively prevented neurite outgrowth out of the channels, forcing the neuron to redirect its growth along the presented substrate (arrows in FIG. 5D).

The TMCC Allows Real-Time Visualization of Axonal Regeneration in vitro

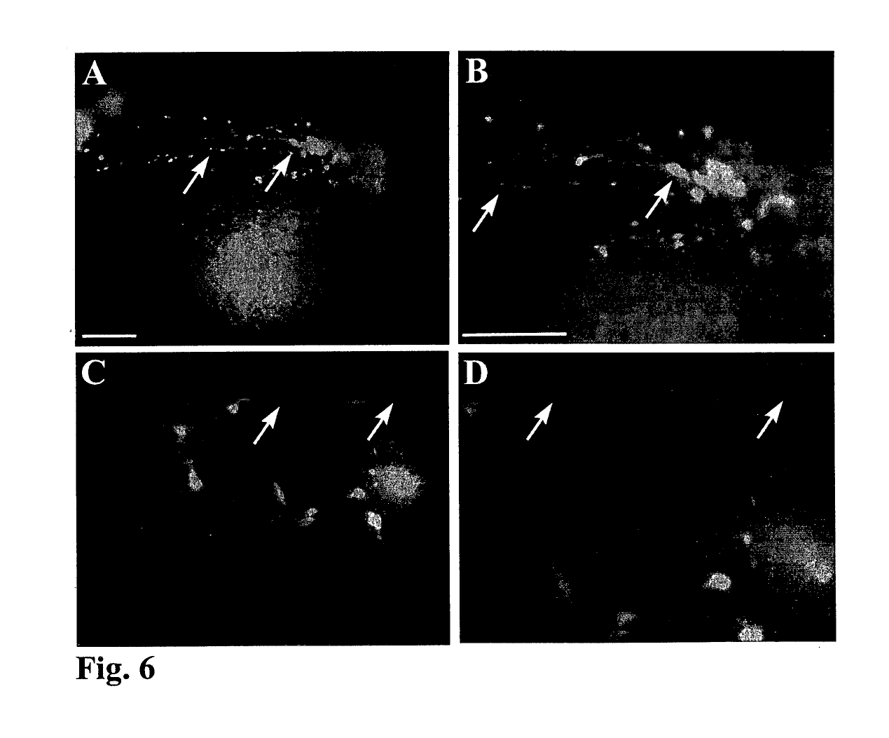

In order to evaluate the capacity of the TMCC to support the growth of DRG explants, as well as its ability to allow the visualization of the regenerative process within the conduit, pieced adult DRGs were placed inside the channels and infected with adenoviral vectors encoding for the green fluorescent protein (GFP-Ad). After 3 days in culture, GFP-expressing neurons were mostly growing within the explant with no neurites elongating inside non-coated channels (FIGS. 6A,B). Two days later, some of the neurons were observed to have extended their axons to the lumen of the channel (FIGS. 6C,D). However, under these conditions, axonal regeneration was limited (≧200 μm in five days) and nonpermanent since growth regression of most extended neurites was observed in the following 48 hrs.

Preseeding of Schwann cells inside the TMCC: Enhanced Directed Axonal Regeneration in vitro

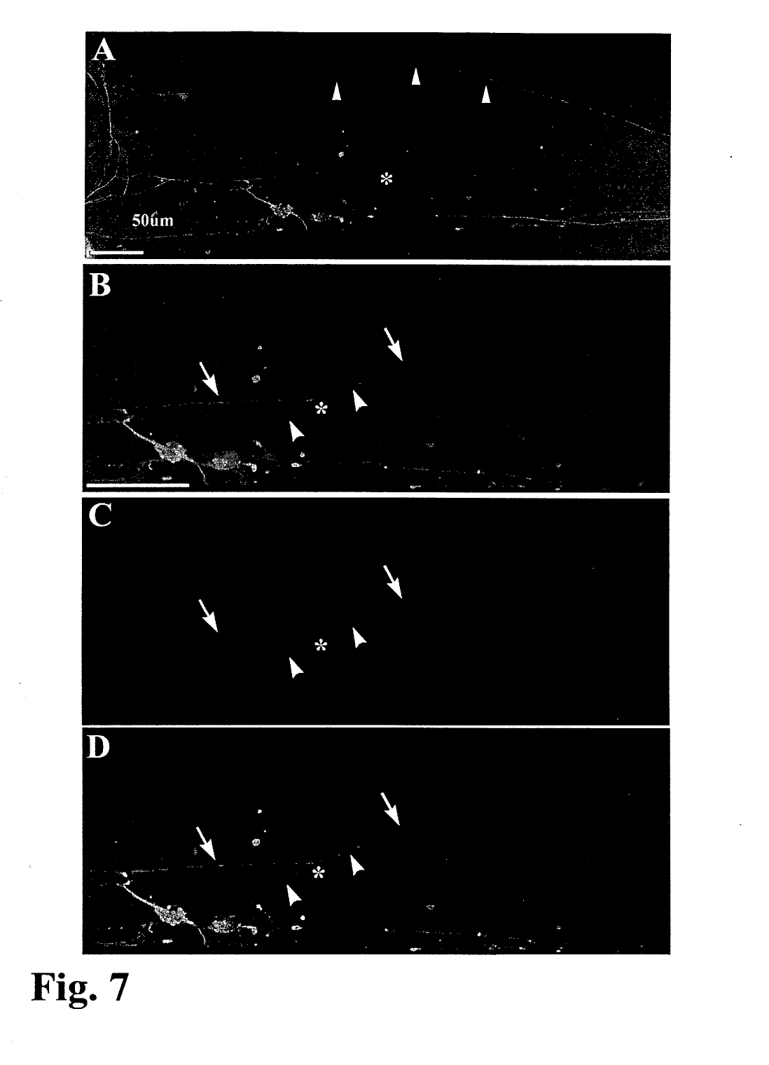

The ability of the TMCC to support the development of Schwann cell cables (FIG. 3C) afforded the opportunity to demonstrate that a preformed substrate of this type of cells should promote and accelerate the regenerative response of DRG neurons. To this end, the channels of a TMCC were seeded with Schwann cells, which were allowed to attach and grow inside the conduit for 12 hrs. At that time, dissociated DRG neurons were loaded inside the channels with and without pre-seeded Schwann cells, and allowed to culture for an additional 24 hrs. As mentioned above, DRG neurons seeded in channels with no Schwann cells grew short neurites along the channels (FIG. 5D). In sharp contrast, conduits with pre-established Schwann cells in the lumen of the conduits showed long neurite outgrowth (1 mm within 2 days). In those cultures, axonal regeneration was clearly directed throughout the lumen of the channels following close contact with Schwann cells. No axonal growth was observed to cross the channel walls or invade other channels (arrowheads in FIG. 7). As demonstrated in FIGS. 7B-D, axonal growth was not only observed to be associated with the Schwann cells, but also to be clearly influenced by them. Axons were observed to travel in one direction (arrows) and to change their trajectory (asterisk) upon encountering a Schwann cell (arrowhead) positioned in that direction.

The transparent cell growth scaffolds of the present disclosure are particularly useful in that cell growth and morphology can be directly visualized to monitor growth and to study the growth of cells under various conditions. The growth characteristics of three separate cultures of cells in the growth channels have been studied. The inventors have demonstrated that the growth conduits remain transparent for at least three weeks allowing long term observation of cell growth. 3T3 cells have been grown in a cluster morphology that is useful in modeling three dimensional tumor growth and in the ability to directly observe the cellular response to various chemotherapeutic agents. In another example of cell growth inside the channels, PC12 cells are shown to grow neurites that are directly observed in the transparent cell scaffold. This embodiment is especially useful in monitoring the growth of neurons for nerve regeneration and repair applications. This observation of cell growth allows the in vitro electrophysiology study of cellular function, and provides a mechanism for the screening of effectiveness of potential drugs, such as antiepileptic drugs.

The use of the present invention for nerve regeneration or tissue engineering is demonstrated by the culture of Schwann cells and mixed cultures of cells in the cell growth conduit. For example, the growth characteristics of Schwann cells in the channels provide the cellular environment to encourage neurite growth. The inventors have demonstrated that Schwann cells, two hours after loading inside the channels, form a natural elongated structure. The expanded and elongated Schwann cell structure can be observed throughout a considerably long segment inside the channel. These studies demonstrate one of the advantages of the invention, in that the cells may be directly observed to monitor growth and formation of a nerve growth channel.

Another advantage of the present invention is the ability to grow and monitor mixed cultures of cells. The inventors have also demonstrated the ability to grow a mixed cellular culture in which, based on cell morphology, fibroblasts and Schwann cells are apparent under phase-contrast microscopy. In addition, mixed cultures of myoblasts and neurons in the cell growth conduits increase the cellular integration between different type of cells, and serve as a tool for the investigation of pathogenesis and clinical diagnosis of various diseases such as neuromuscular disorders.

One advantage of the cell conduits of the present disclosure is the ability to load cells in various combinations of cell types and densities for a variety of uses. One such advantage is offered by the ability to load cells in a cell density gradient from a proximal end of a conduit to a distal end. In another embodiment of the transparent cell growth channels, a multichannel conduit can be loaded either at different cellular densities in different channels, or with the same cell concentration in all channels within a conduit or scaffold.

### EXAMPLE 2

Transparent Multi-Channel Matrix (TMM)

Design: The present example describes improvements to the original design of the templates and multichannel devices. These improvements provide a casting mold that allows one to precisely define the inter-fiber distance and the parallel and horizontal distribution of the fibers used for casting of the multiple channels within the matrix. This improved design provides certain advantages over the currently practiced 3-D cell culture technology, including allowing for (1) the seeding of selected cell lines into specific and separate channels and (2) the separate seeding of different cells into the same channel, these capabilities are unique to the TMM.

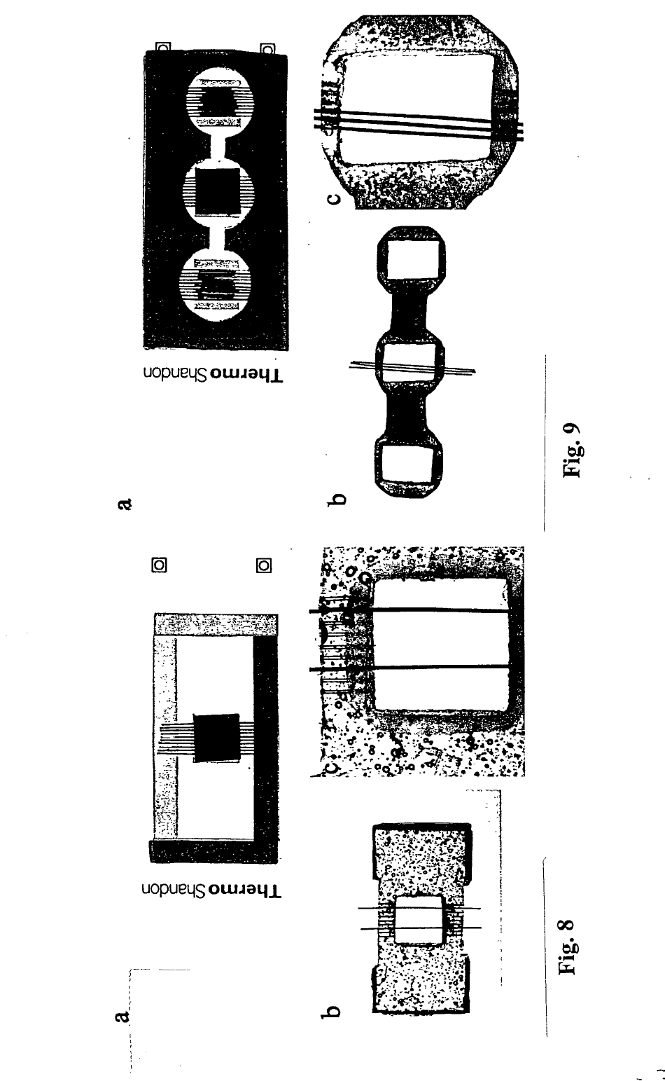

Polymer prototype molds were designed with the capacity to cast either one (FIG. 8) or three (FIG. 9) gels simultaneously. For both prototypes the method of making the mold (FIGS. 8A, 9A) as well as the final cast are shown (4×; FIGS. 8B, 9B, 10×; FIGS. 8C, 9C). Both prototypes allow for the casting of multiple, equidistant, parallel channels. The method used in the making of the prototypes and their application in the casting of the TMM, are described bellow.

Material and Methods. Under the stereomicroscope, dental wax, with a thickness of 1.25 mm, was sectioned into several 8 mm squares to form the central fiber-support cast and into 4 mm strips to form the perimeter of the prototype shown in FIG. 8 and a 45 mm×22 mm rectangle with three 11 mm diameter holes spaced 4 mm apart to form the multicasting prototype shown in FIG. 9. To form the base layer, an 8 mm square of wax was centered onto a 75 mm×25 mm glass microscope slide, applying slight pressure to secure it in place. The 4 mm perimeter wax strips forming the long axis were then positioned at a distance of 2.5 mm and the strips forming the short axis at 32 mm from the central wax square. Petroleum jelly was lightly brushed onto the surface of the central square to aid in the positioning of the synthetic fibers. Fibers, 18 mm×0.25 mm were then placed onto the central square with the ends overlapping onto the perimeter strips. Small 6 mm fiber sections were placed between each long fiber to achieve a uniform distance between them-these did not extend beyond the edges of the 8 mm central square. After parallel positioning of approximately ten long fibers, pressure was applied to embed them into the wax and secure them in the desired position. Petroleum jelly was lightly brushed onto the floor of the glass slide and the section of the long fibers that was going to be exposed to the orthodontic resin to keep the resin from adhering to their surfaces. A cap layer of dental wax was applied onto the base layer and pressure applied to secure it in place. In this way, the synthetic fibers were sandwiched between the two layers of wax, insuring their position in a uniform horizontal plane with a distance of 0.25 mm between each fiber. Orthodontic resin liquid and powder were then mixed to a liquid consistency and poured into the wax cast. When the resin had hardened, the perimeter wax and the central cap layer were removed to expose the synthetic casting fibers. Using forceps, the fibers were then extracted from the resin cast and the remaining wax removed. The resulting channel matrix-casting prototype device was then washed with warm water and detergent followed with a wash in 100% ethanol to remove any residual petroleum jelly or wax.

To cast a channel gel matrix, long synthetic fibers were reinserted into the holes cast into the resin prototype device and this was then placed onto a clean microscope slide. Agarose at a concentration of 1.5-5.0% was prepared and poured into the mold. The agarose was placed at 4° C. to cool into a gel state; the synthetic fibers were then removed, producing channels within the gel matrix. The resulting channels were then ready for cell seeding. A multi-matrix casting device was also constructed in the manner described above. This allowed for an increase in productivity. NOTE: all materials were sterile and the procedure was done under sterile conditions in a laminar flow hood.

Direct Cell Placement within a Complex Multicellular 3-D Culture

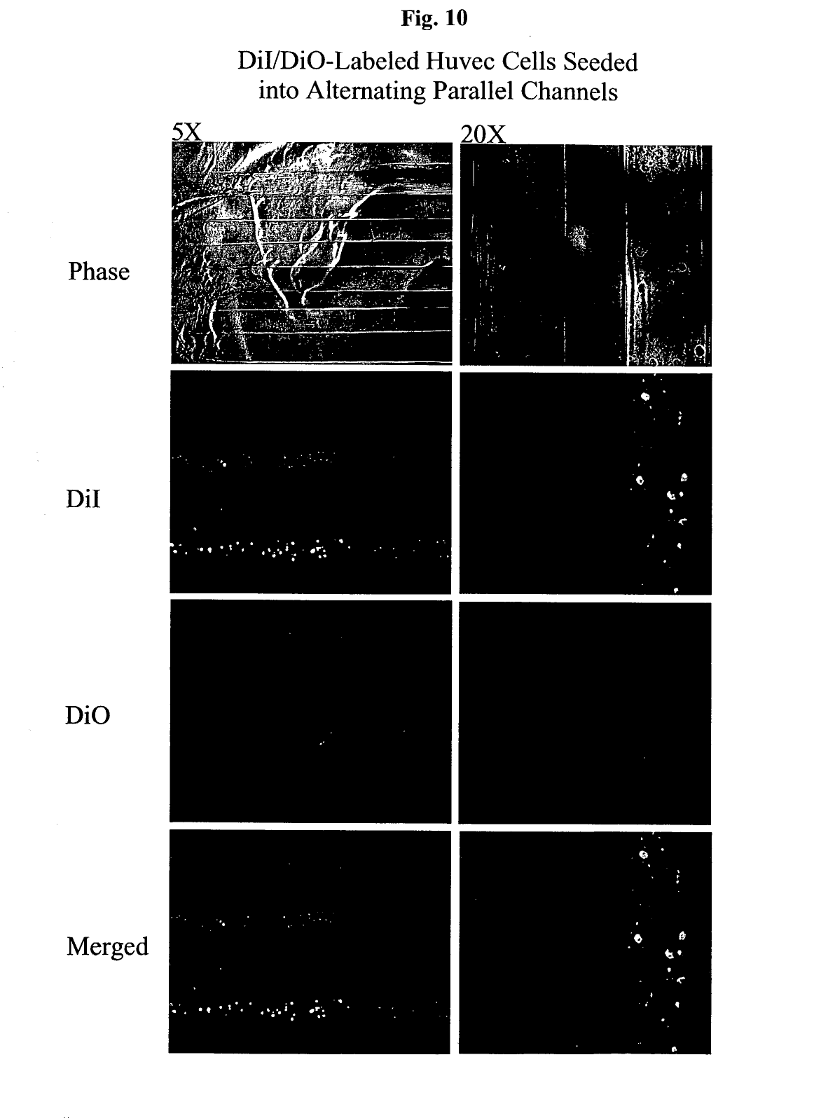

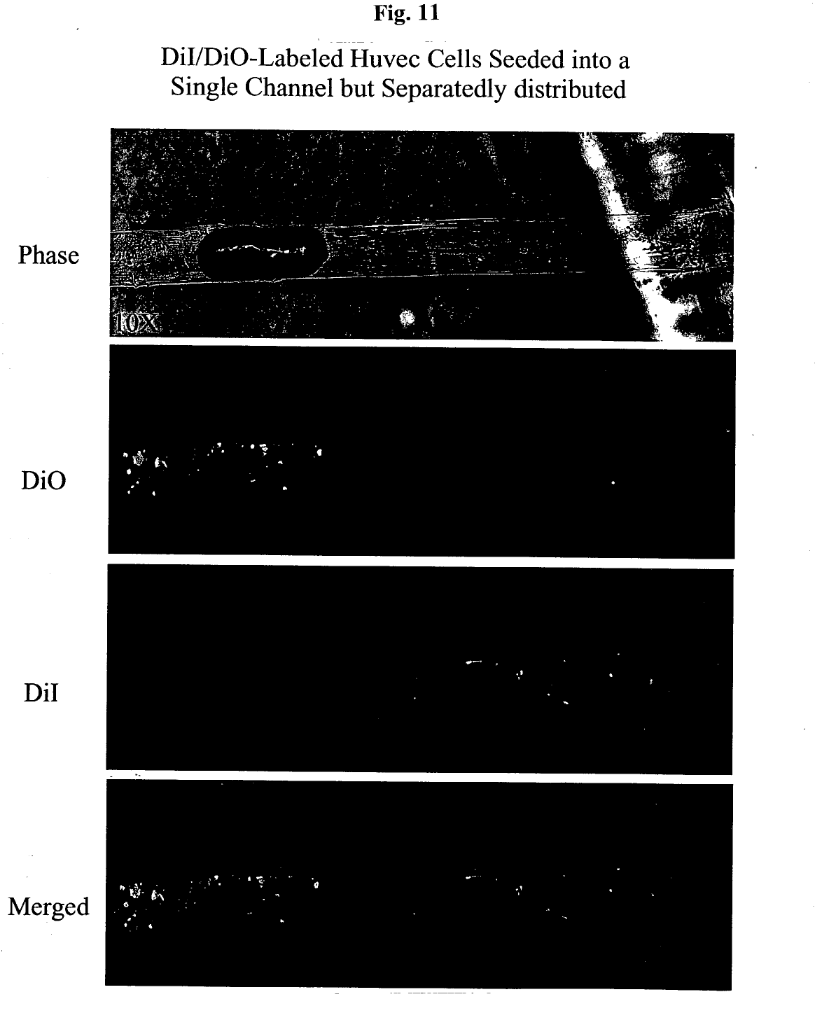

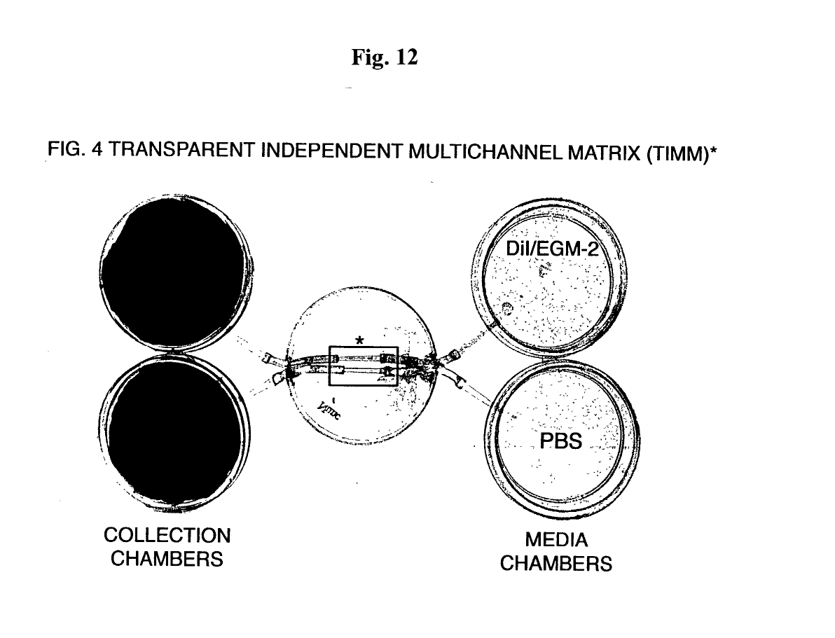

Current 3-D cell culture technology does not allow the user to choose a particular place within the matrix for specific cell types, while setting up a complex multicellular culture. In contrast, the individual channels within the TMM are separately accessible for cell seeding, making it possible to specify the placement of separate cell types within the 3-D matrix as shown herein. Human umbilical vein endothelial cells (HUVEC) cells were cultured in separate 2-D 35 mm culture dishes, and 24 hrs after plating the cells were incubated for 15 min with media containing 0.5% w/v of either a red (DiI) or a green (DiO) lipophilic dye, in order to label the separate cell populations. The cells were then dissociated by trypsinization and loaded into a 10 ul Hamilton syringe. The syringe was secured onto a micromanipulator and with the aid of a Zeiss STEMI 2000-C stereomicroscope the DiI- and DiO-labeled cells were seeded into separate channels within the matrix. DiI- DiO-labeled HUVEC cells were also seeded into the channels by a combination of capillary action and the alternated removal of selected fibers. Selective fiber removal allowed for loading the channels with a cell/ECM (extracellular matrix) gel mixture, the cell/ECM gel mixture was allowed to polymerize by incubation for 5 min at 37° C. This allowed the seeding of differentially labeled cell types at later time points into other channels by the removal of a casting fiber. Using this last approach alternating channels were seeded with DiI (red fluorescing) and DiO (green fluorescing) labeled HUVEC cells, as clearly demonstrated in FIG. 10.

The devices of the present disclosure also enable the loading of two different cells into the same channel as shown in FIG. 12. In this procedure, after polymerization of the agarose gel in the casting chambers, the plastic fibers used to cast the individual channels were partially removed from the gel, thus opening only half of the channel for cell loading. The DiI/ECM mixture was then loaded by capillarity. That is, the cells were placed into the side of the gell containing the opened half, and the cells were drawn inside by capillarity using a filter paper applied to the opposite end of the gel. When the DiI cells were in place, the gel was incubated at 37° C. for polymerization of the extracellular matrix gel for 5 minutes. This step effectively immobilizes the DiI cells in that half of the channel. The fibers were then completely removed, thus opening the other half of the channel for cell loading with a different population of cells. The seeding of the DiO cells was then achieved by repeating the same procedure as described for the first end of the gel.

The seeded channel matrices were incubated in a 35 mm culture dish containing EGM-2 media (Biowhittaker), and observed for a period of more than 15 days. During the course of this time, the labeled HUVEC cells attached, differentiated and proliferated within the channels of the TMM. The use of the TMM thus provides a unique 3-D model for cell culture that is not possible by current methods.

### EXAMPLE 3

Cancer Research: Bystander effects. The TMM model allows for a better evaluation of “bystander effects” (the killing of normal cells) that results from pharmacological and gene therapies used in cancer treatments. The study of bystander effects is currently limited by the lack of physiologically relevant experimental models. The TMM offers clear advantages over other 3-D culture systems in this regard, since individual channels serve as ways to separate normal cells from tumor cells, therefore, allowing a real time follow-up of the effects of cancer therapies on both tumor as well as normal cells. In one method of use, a TMM is seeded with normal cells and glioma cells in alternating, separate channels, with the glioma cells transfected with a pro drug-activating suicide gene such as cytosine deaminase.

By bathing the culture with 5-fluorocytosine, the transfected cells release cytotoxic metabolites that diffuse through the matrix to channels containing normal cells, thus killing them. In this regard, the TMM is used as a screen for the “bystander effects” of potential cancer treatments. Furthermore, the ability to cast agarose gels with varying pore sizes, depending upon the concentration of the preparation, allows the agarose matrix to function as a molecular sieve between the channels. This provides information on whether or not a particular treatment produces a “bystander effect”, and also provides information on the molecular size of the toxic metabolite, aiding in the identification of these compounds.

### EXAMPLE 4

Tissue Engineering, Artificial Organs and Gene Therapy

Engineered tissues involve the seeding of biodegradable scaffolds with donor cells to induce controlled cell growth in vitro, which are then used in implants. Examples of such implanted tissues include skin, cartilage, bone, vasculature, heart, liver, and nerve. Vascularization is one of the most important requirements for tissue-engineered organs. Since the nature of the TMM allows for the selective growth of human vascular endothelial cells in specific 3-D channels, this feature provides the framework for the design of multicellular bioartificial organs. In the TMM, some channels are used to seed specific cell subtypes of a particular organ, and other channels are seeded with vascular endothelial cells to provide for proper vascularization. In contrast to the currently available technology, the flexibility in the design of the TMM allows for the possibility of culturing multiple cell types in both a temporal and spatially controlled manner.

Cartilage and Bone Tissue Culture

Considerable effort is being made in the field to discover methods for regenerating and repairing bone and cartilage. The need for bone substitutes is particularly important since approximately 500,000 surgical procedures are performed every year in the U.S. for various conditions including trauma, congenital and degenerative diseases, cancer and cosmetics production. Several techniques, including bone implants and grafts are showing promise for providing a remedy for skeletal disorders and chondrodystrophies. The TMM creates a 3-D culture environment conducive to cell aggregation, and provides a powerful new tool for the dynamic study of bone formation and chondronic mutations.

In addition, The TMM offers the capability of using both plasmid and viral DNA transfection agents to conduct gene expression studies. Also, the casting device here disclosed could be used with other synthetic polymer systems for the scaffolding of other tissues and organs.

### EXAMPLE 5

Transparent Independent Multi-Channel Matrix (TIMM)

Design: A further improvement was made in the basic design of the disclosed devices in order to facilitate pharmacological and genetic screens. This improved design, called the Transparent Independent Multi-channel Matrix (TIMM) by the inventors, provides direct cell placement and independent media perfusion into each channel in the TMM. In this way, every channel is able to serve to test separate genetic, molecular, or pharmacological interactions, thus enabling the testing of multiple agents or the testing of the reaction to a single agent by multiple cell populations in a single step. The improved culture device is composed of separate chambers for culture media for each channel, as well as respective separate media collection compartments, with a controlled flow rate. The channels are isolated by casting a channel within the matrix inside an impermeable tube of slightly larger diameter. The tubing is then, separately connected to the media and media collection chambers packed with absorbent material for independent media perfusion. An example of such a device used in the isolation of individual channels is shown in FIG. 12.

Material and Methods

A 0.25 mm diameter section of stainless steel wire was centered in a section of transparent, synthetic, tubing with an internal diameter of approximately 1 mm×15 mm long. Agarose was then injected into the transparent tubing and allowed to gel at 4° C. The steel wire was then removed and the result was a transparent tube coated internally with agarose, with a central channel for cell seeding. A device was then fabricated using five 35 mm culture dishes. The TIMM was placed side-by-side and secured to the floor of a central 35 mm culture dish. Small internal diameter tubing was then attached to each end of the TIMM. One end of the tube was attached to a micropipette tip and inserted into a small hole pierced at the level of the floor of a 35 mm culture dish containing media and the other end, also attached to a micropipette tip, was inserted, at midlevel, into a 35 mm culture dish containing absorbent material for media collection. This was done to establish a separate, individual, unidirectional flow, via capillary action within each TIMM.

A further study was done to demonstrate the ability of the TIMM to separately perfuse independent channels. Using a slow (approximately 0.05 ml/min) flow rate, two contiguous, yet separate channels were placed on the TMM device. One channel was perfused with saline only, while the other was perfused with media containing 0.5% of DiI crystals. Pictures taken at a dynamic flow rate showed that only one of the channels was perfused with the DiI solution and that the dye did not diffuse from it. At higher magnification (10×) only the DiI perfused channel was shown to better demonstrate the flow of red fluorescent crystals in that channel. Because of the exposure time, the camera did not match the speed at which the crystals passed through the channel. DiI pictures taken at dynamic flow rates showed diffuse fluorescence. In contrast, pictures taken from a separate insulated channel (the tube diameter is slightly increased over that used for the dynamic flow rate pictures, but the internal diameter of the channel remains the same) perfused with the same DiI solution, but with a passive flow rate, weres shown for comparison, to demonstrate that in the absence of a dynamic flow rate, the DiI crystals remain immobile and are clearly photographed. The demonstrated ability of the TIMM to separately perfuse individual channels, demonstrates that this device allows for the complete isolation of media conditions in each channel and that this unique property can then be exploited for the screening of genetic, molecular and pharmacological interactions in 3-D cultures. The following examples are studies that are enabled by the TIMM.

### EXAMPLE 6

Use of TIMM as a Bioreactor for Cell Growth and Antibody Production

Hollow-Fiber Cell Culture is a method that utilizes hollow fibers to create a semi-permeable barrier between the cell growth chamber and the medium flow. The molecular weight cut off of the hollow fiber is used to trap secreted proteins or other cellular products in the small volume of the extra-capillary space. This results in concentrations up to 100 times greater than can be achieved in roller bottle or flask cell culture.

Antibody production efficiency. It is contemplated by the present inventors that the TIMM device offers certain advantages over conventional antibody production using hollow-fiber cartridges. Bioreactors with a low glucose utilization rate (GUR) have too few metabolically active cells. Conversely, a high GUR may lead to rapid overgrowth and premature senescence. The TIMM is expected to be able to accommodate the growth of more dense cellular cultures per area unit. To confirm this, the rates of antibody production and cell growth in conventional hollow-fiber cartridges are compared to those of the TIMM. Both the number of days required to reach a target GUR level of 250 mg/hr, as well as the number of days required to produce 1 g of antibody per volume of the feed medium consumed (production efficiency) are determined. Hollow-fiber bioreactor cartridges using the Cellex, Inc., Acusyst Jr. and the Unisyn Technologies, Inc., have been reported to display lengthened lag phase in antibody production and overall antibody production efficiency. In conventional hollow-fiber systems with continuous mode of harvest, a peristaltic pump is used to withdraw medium from the extracellular space at a slow, constant rate (up to 3.5 ml/min). Since the preliminary studies with TIMM have indicated that flow rates of 0.05 to 0.1 ml/min can be achieved, this device has the potential to produce more antibody per liter of medium consumed. Because of the small volumes of media used by the TIM, harvesting the antibody at such slow rates rather than at a constant higher rate is expected to increase the concentration of the antibody produced.

Cell Removal. The removal of cells from confluent hollow-fiber cartridges is useful to maintain the antibody production level of the bioreactor run. Routinely, cells are removed from the cartridge by attaching syringes filled with media to the extracapillary ports and vigorously pushing and pulling the plunger of the syringes. This cell-removal procedure leaves large numbers of dead cells and cellular debris within the cartridge, thus affecting subsequent cellular growth and antibody production within the cartridge. Conversely, the TIMM offers the advantage of the ability to exchange the cell culture compartment with one containing pre-seeded cells, reducing time and optimizing resources. The TIMM, therefore, offers a more efficient alternative for 3-D tissue culture and antibody production.

Cancer Research: Pharmacological and Genetic Screens in 3-D Culture Systems

Classical culture methods for malignant cells include culture plates and microcarrier particles. In these configurations tumor cells rarely grow to more than a confluent monolayer, which is far from resembling their natural growth pattern, a three-dimensional tissue mass. TIMM is used to grow, and study in real time, malignant changes of cancers as they develop from single cells to 3-D tumors. Various tumor types may be studied, including but not limited to melanoma, prostate cancer, breast cancer, ovarian cancer, osteosarcoma, glioma, and colon cancers, as examples. TIMM provides additional advantages, compared to current technology, in the examination of gene expression as a function of the stage of tumor cell aggregate growth. The ability to seed different cells in different channels within the TIMM allows for experimental designs in which both pharmacological and genetic screens are used for the identification of genetic makers for patient diagnosis and in the design of specific molecular therapies. In one example of the utility of the disclosed devices, one can seed 10-100 different tumor cell lines in corresponding separate channels, with simultaneous treatment with drugs that would activate capsase-3, a proapoptotic marker. If the green fluorescent protein (GFP) is used to label the activated form of capsase-3, then, one would be able to identify in real time those cells that are undergoing apoptosis as a result of a particular treatment. TIMM thus provides the ability to detect drug effects on a single cell in a population within a single channel, as well as effects on isolated cell populations separated into up to 100 different channels. It is also contemplated that microlithographic laser technologies can be utilized to create models of the TIMM devices with 1000 or more channels, thus allowing high throughput screening for drug discovery.

This example of use may also be applied to screening for cells responsive or resistant to particular drugs. An important diagnostic tool includes the use of the TIMM in the testing of tumor cells from a particular patient in a pre-screening test for drug sensitivity prior to chemotherapy, or as a step in determining genotypic or proteomic characteristics of drug sensitivity or resistance.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A transparent agarose cell scaffold comprising a plurality of cell growth channels contained in the scaffold, wherein the scaffold is configured such that biological cells loaded into the channels are observable by microscopy during growth of the cells in the channels.

2. A transparent agarose cell scaffold according to claim 1, wherein the scaffold is substantially cylindrical in shape.

3. A transparent agarose cell scaffold according to claim 1, wherein the scaffold is substantially rectangular or square in shape.

4. A transparent agarose cell scaffold according to claim 1, wherein the channels extend the entire length of the scaffold effective to provide a liquid conduit from one end of the scaffold to the other end of the scaffold.

5. A transparent agarose cell scaffold according to claim 1, wherein the channels are formed in the body by removing elongated members from the agarose body after gelling.

6. A transparent agarose cell scaffold according to claim 1, wherein the channels are formed of transparent hollow tubes with an interior coating of agarose.

7. A transparent agarose cell scaffold according to claim 1, wherein the channels are loaded with biological cells.

8. A transparent agarose cell scaffold according to claim 7, wherein the cells are prokaryotic, eukaryotic, protozoan or fungi.

9. A transparent agarose cell scaffold according to claim 7, wherein the cells are human cells.

10. A transparent agarose cell scaffold according to claim 7, wherein the cells are glial cells, endothelial cells, muscle cells, or neurons.

11. A transparent agarose cell scaffold according to claim 7, wherein cells are loaded in a channel such that a density gradient of cells is formed along at least a portion of a channel.

12. A transparent agarose cell scaffold according to claim 7, wherein two or more cell types are loaded into the channels.

13. A transparent agarose cell scaffold according to claim 12, wherein two or more cell types are loaded into a single channel.

14. A transparent agarose cell scaffold according to claim 12, wherein a first channel is loaded with a first type of cell and a second channel is loaded with a second type of cell and wherein the first and second types of cells are different.

15. A transparent agarose cell scaffold according to claim 7 wherein the cells are Schwann cells, myoblasts, 3T3 cells, PC 12 cells, NG108 cells, astrocytes, oligodendrocytes, fibroblasts, endothelia cells, chondrocytes, osteoblasts, or stem cells.

16. A transparent agarose cell scaffold according to claim 7, wherein the cells express cell growth factors.

17. A transparent agarose cell scaffold according to claim 16, wherein the cells are engineered to express one or more growth factors.

18. A transparent agarose cell scaffold according to claim 16, wherein the cells express nerve growth factor (NGF), ciliary neurotrophic factor (CNTF), glial cell line derived neurotrophic factor (GDNF), brain-derived-neurotrophic factor (BDNF), neurotrophin-4/5 (NT-4/5), neurotrophin-3 (NT-3), insulin-like growth factor (IGF-I), basic fibroblast growth factor (FGF-2), leukemia inhibitory factor (LIF), transforming growth factors (TGFs)-β, GGF, or a combination thereof.

19. A transparent agarose cell scaffold according to claim 16, wherein the cells express one or more cell adhesion molecules.

20. A transparent agarose cell scaffold according to claim 16, wherein the cells express L1, N-CAM, NgCAM/L1, L2/HNK-1, N-cadherin or a combination thereof.

21. A transparent agarose cell scaffold according to claim 16, wherein the cells express one or more extracellular matrix molecules.

22. A transparent agarose cell scaffold according to claim 16, wherein the cells express laminin, heparan sulphate proteoglycans (HSP), tenascin, fibronectin or a combination thereof.

23. A transparent agarose cell scaffold according to claim 16, wherein the cells express one or more chemoattractants or chemorepellents.

24. A transparent agarose cell scaffold according to claim 16, wherein the cells express Slit, Netrin, Ephrins, NOGO-A, MAG, CSPGs, semaphorins, or collapsing.

25. A transparent agarose cell scaffold according to claim 2, wherein the scaffold has an outside diameter of about 0.5 to about 3 mm.

26. A transparent agarose cell scaffold according to claim 3, wherein the agarose body has a thickness of from about 0.25 mm to about 3 mm.

27. A transparent agarose cell scaffold according to claim 1, wherein the channels have a diameter of from about 0.06 mm to about 0.2 mm.

28. A transparent agarose cell scaffold according to claim 1, wherein the channels have a diameter of about 0.17 mm.

29. A transparent agarose cell scaffold according to claim 6, wherein at least one channel is connected at a first end to a source of media and to a second end to an absorbent material effective to flow medium into the channel by capillary action.

30. A transparent agarose cell scaffold according to claim 29, wherein a plurality of channels are connected at a first end to separate sources of medium.

31. A method of constructing a cell growth scaffold comprising:
forming an agarose body having a first end and a second end, and including elongated members embedded in said agarose body and extending to at least the first or second end thereof, and
removing at least one of said elongated members when the agarose body is in a solid state effective to form a channel in the substantially solid agarose body,
wherein the body is substantially transparent such that cells seeded into the channel during use are visible by microscopy techniques.

32. The method of claim 31, wherein the solid agarose body comprises multiple channels extending from the first end to the second end, wherein the channels provide a fluid connection through the body from the first end to the second end of the body.

33. The method according to claim 31, wherein the agarose concentration is from about 0.5% to about 5.0%.

34. The method according to claim 31, wherein the body is substantially cylindrical and has an outside diameter of about 0.5 to about 3 mm.

35. The method according to claim 31, wherein the body is substantially rectangular or square.

36. The method according to claim 35, wherein the agarose body has a thickness of from about 0.25 mm to about 3 mm.

37. The method according to claim 31, wherein the channels are formed by a fiber or wire that has diameter of from about 0.06 mm to about 0.17 mm.

38. The method according to claim 31, wherein the body comprises from about 1 to about 100 channels.

39. The method according to claim 31, wherein the elongated members are formed by:
placing a wire or fiber in a transparent hollow tube;
pouring melted agarose into the tube around the wire or fiber and allowing the agarose to gel; and
removing the wire or fiber to obtain cell growth channel comprising a hollow tube with an internal coating of agarose.

40. The method according to claim 31, wherein Schwann cells are grown in the channels to provide a scaffold for nerve regeneration.

41. The method according to claim 31, wherein cells are grown in the channels as a scaffold for tissue engineering of muscle, tendon, blood vessel, pancreas or liver tissue.

42. A method for screening a candidate substance for an effect on the growth, motility or viability of a target cell comprising:
providing a cell scaffold of claim 1 in which one or more channels are loaded with the target cells;
contacting the target cells in the channels with the candidate substance;
observing the contacted cells microscopically;
comparing the contacted cells to identical control cells in the absence of the candidate substance;
wherein a difference in the growth, motility or viability of the contacted cells relative to the identical control cells is indicative of an agent that affects the growth, motility or viability of the target cells.

43. The method according to claim 42, wherein the target cells comprise Schwann cells, myoblasts, 3T3 cells, PC 12 cells, NG108 cells, astrocytes, oligodendrocytes, fibroblasts, endothelia cells, chondrocytes, osteoblasts, or stem cells.

44. The method according to claim 42, wherein the cells are Schawnn cells loaded into channels coated with extracellular matrix gel.

45. The method according to claim 42, wherein the agarose gel comprises pores, permitting nutrients and gas to pass through the pore of the conduit but inhibiting neurite growth through the pores.

46. The method according to claim 42, wherein two or more cell types are loaded into a single channel.

47. The method according to claim 42, wherein two or more cell types are loaded into individual channels.

48. The method according to claim 42, wherein cells are seeded in a density gradient within a channel.

49. The method according to claim 42, wherein cells genetically engineered to secrete nerve growth factors are loaded into the channels in a gradient pattern to promote the directional growth of nerve regeneration.

50. The method according to claim 40, wherein cells genetically engineered to secrete nerve growth factors are loaded into the channels in a gradient pattern to promote the directional growth of nerve regeneration.

51. The method according to claim 42, wherein the two or more populations of cells are inducible for differential expression of one or more gene products.
